(19) United States
(12) Patent Application Publication
(10) Pub. No.: US 2012/0276167 A1
(43) Pub. Date: Nov. 1, 2012

(54) IMMUNOPROTECTION BY ORAL ADMINISTRATION OF RECOMBINANT LACTOCOCCUS LACTIS MINI-CAPSULES

(75) Inventors: Dominic Man-Kit Lam, Hong Kong (CN); Yuhong Xu, Hong Kong (CN)

(73) Assignee: VAXGENE CORPORATION, Hong Kong (CN)

(21) Appl. No.: 13/518,592

(22) PCT Filed: Dec. 23, 2010
(86) PCT No.: PCT/US2010/062034

§ 371 Date: Jun. 22, 2012

Related U.S. Application Data
(63) Continuation-in-part of application No. PCT/US2010/041792, filed on Jul. 13, 2010.
(60) Provisional application No. 61/289,663, filed on Dec. 23, 2009.
(60) Provisional application No. 61/224,973, filed on Jul. 13, 2009.
(60) Provisional application No. 61/263,215, filed on Nov. 20, 2009.

(51) Int. Cl. A61K 39/145; A61P 31/16; A61K 9/00

(52) U.S. Cl. 424/400; 424/200.1

(57) ABSTRACT

In one embodiment, the present invention provides for an edible mini-capsule form of live, non-persisting, recombinant Lactococcus lactis (L. lactis) vaccine against a pathogen such as the highly virulent influenza H5N1 strain. Enteric coated capsule of the present invention induced high levels of hemagglutinin-specific serum IgG and fecal IgA antibody production after oral administration in mice and chickens, and rendered complete protection against a lethal challenge of H5N1 virus in mice. The present invention thus demonstrates a broadly applicable platform technology for producing and administering edible vaccines against bacterial and viral infections.

This application claims the benefit of U.S. Ser. No. 61/289,663, filed Dec. 23, 2009, and claims the benefit of priority of Int'l App'l No. PCT/US2010/041792, filed Jul. 13, 2010, the entire contents and disclosures of which are incorporated by reference into this application.

### FIELD OF THE INVENTION

This invention relates generally to vaccinations. In one embodiment, the present invention provides compositions and methods of using genetically modified Lactococcus lactis strains as an oral vaccine.

### BACKGROUND OF THE INVENTION

The highly pathogenic avian influenza H5N1 virus is considered a great threat to worldwide human and animal health. This virus strain is highly susceptible to antigen drift and has already caused several outbreaks in human subjects with very high mortality rate (1). Vaccination is considered the most desirable counteraction to prevent the spreading and rapid mutation of the virus. It is also highly preferable to develop vaccines for all the species affected to slow down cross-species spreading. However, conventional influenza vaccines made of inactivated viruses could hardly be useful for the H5N1 strain because of difficulties in manufacturing and the general requirement of multiple injections to every subject (2, 3). New vaccine preparations, including various subunit vaccines (4), DNA vaccines (5,6) and recombinant adenovirus vaccines (7,8) are being examined, but they all require injection which would be impossible for wild birds and costly and troublesome for humans and farm animals. In this regard, safe and efficacious oral vaccines would be ideal since they can be added to the food or drinks of the subjects to be immunized.

There has been a long pursuit of edible vaccines that are safe, effective and convenient to use. The use of edible plants such as tomato and potato to express antigens that could be taken orally for the vaccination of human and livestock animals has been previously proposed (9-11). Recently, rice was used as an antigen expression and delivery vehicle and showed to convey immunity against cholera in mice (12). The use of edible plants as expression and delivery vehicles for vaccines offer many advantages for a variety of infectious diseases, especially those whose pathogens are relatively conserved. This is because it normally takes over 1 year to produce stably expressed transgenic plants. In contrast, for diseases in which the infective viruses mutate or shift rapidly, such as the influenza viruses, expression vehicles that are both edible and can be transformed in a matter of weeks are much more desirable. It is mainly for this reason that the lactic acid bacteria (LAB) was chosen: it is generally regarded as safe (GRAS) and widely consumed or used in food products. This approach has been reported in studies using recombinant L. lactis encoding various antigens for oral administration (13-16), resulting in a variety of immune responses thought to be related to the type of antigen, the amount of antigen expressed, and the duration of antigen expression in the gut (17, 18).

### SUMMARY OF THE INVENTION

The development of safe and efficient influenza vaccines for human and animal use is essential for preventing virulent outbreaks and pandemics worldwide. In the present invention, it is found that genetically modified lactic acid bacteria such as Lactococcus lactis strains expressing the avian influenza HA gene can be used as an oral vaccine for the protection of H5N1 virus infection.

In this study, several influenza H5N1 hemagglutinin (HA) antigen expression vectors were constructed based on a well engineered nisinA-induced L. lactis expression strain. They either expressed the antigen in the cytoplasm (L2), or secreted the antigens (L3), or displayed the antigens on the cell wall (L4). In one embodiment, these vectors were formulated with mucoadhesive polymers and surface enteric coating. After oral administration of the enteric-coated antigen displayed expression vector and antigen secreted expression vector respectively, the resulted immune responses were greatly improved, resulting in complete protection of the immunized mice from a lethal dose of viral challenge.

In one embodiment, oral administration of genetically modified Lactococcus lactis strains disclosed herein induced strong HA-specific humoral and mucosal immune responses in subjects which were able to withstand lethal dose of H5N1 virus infection.

### BRIEF DESCRIPTION OF THE FIGURES

FIG. 1 shows western blot analysis of recombinant L. lacti vectors (A, B and C), and immunofluorescence analysis of L4 (D) (X200).

FIG. 2 shows HA-specific antibody titers detected by ELISA. Mice (2A and 2B) were orally immunized with enteric capsules and non-enteric live bacteria. (A) HA-specific serum IgG was determined by ABS-ELISA using recombinant HA protein as a coating antigen. (B) HA-specific mucosal IgA was determined from the fecal pellets. (C) Chickens were immunized orally or subcutaneously with L4. Chickens sera IgG were assayed by ABS-ELISA. *Represents statistically significant differences relative to the PBS, L1 and capsule-L1 controls. (*p<0.05). Data are given as mean ±SD of duplicate experiments.

FIG. 3 shows cell-mediated immune responses induced by enteric coated recombinant L. lactis. * Represents statistically significant differences relative to the PBS, L1 and capsule-L1 controls. Data are represented as mean ±SD of triplicate experiments.

FIG. 4 shows immune protection against H5N1 virus lethal challenges after oral deliveries of different vaccine preparations. Mice were infected intranasally with H5N1′ virus 2 weeks after the last immunization. (A) Mean weight loss (%) of mice 6 days after infection. (B) Percent survival of mice 0-14 days after infection. n=5 mice per group.

### DETAILED DESCRIPTION OF THE INVENTION

The following terms shall be used to describe the present invention. In the absence of a specific definition set forth herein, the terms used to describe the present invention shall be given their common meaning as understood by those of ordinary skill in the art.

As used herein, the term “edible vaccines” refers to vaccine formulations that are administered and effective via the peroral route.

As used herein, the term “protective immune responses” refers to immune responses resulted from vaccine administrations that can protect the animal from death when challenged with 10 times lethal dose.

As used herein, the term “heterologous antigen” refers to antigens from heterogeneous pathogens such as viruses or bacteria.

As used herein, the term “mucoadhesive polymers” refers to synthetic or natural polymers that interact with the mucus layer covering the mucosal epithelial surface and mucin constituting a major part of the mucus. Recently, a drug delivery system is developed with the use of mucoadhesive polymers that will attach to related tissue or to the surface coating of the tissue for the targeting of various absorptive mucosa such as ocular, nasal, pulmonary, buccal, vaginal etc. This system of drug delivery is called mucoadhesive drug delivery system. Such drug delivery system could be used to deliver the therapeutic agent of the present invention. Naturally occurring mucoadhesive polymers include, but are not limited to, hyaluronic acid and chitosan.

Drug delivery using mucoadhesive dosage form via transmucosal route bypasses hepato-gastrointestinal first pass elimination associated with oral administration, thereby increases the bioavailability and produces longer therapeutic effect. In general, there are two broad classes of mucoadhesive polymers: hydrophilic polymer and hydrogels. Hydrophilic polymers containing carboxylic groups exhibit very good mucoadhesive properties, representative examples include, but are not limited to, poly vinyl pyrrolidone (PVP), methyl cellulose (MC), sodium carboxy methylcellulose (SCMC), hydroxy propyl cellulose (HPC) and other cellulose derivative. Hyrogels are the class of polymeric biomaterials that swell by absorbing water through adhesion with the mucus that covers epithelia. In general, hydrogels are a class of polymer materials that can absorb large amounts of water without dissolving due to physical or chemical crosslinkage of hydrophilic polymer chains. One of ordinary skill in the art would readily construct hydrogels from various well-known monomers, prepolymers or existing hydrophilic polymers. Polymeric biomaterials useful for hydrogel formation usually possess anionic groups (e.g. polyacrylates and their crosslinked modifications, etc) and/or cationic groups (such as chitosan and its derivatives).

For bioadhesion or mucoadhesion to occur, a succession of phenomena is required. The first stage involves an intimate contact between a mucoadhesive and a membrane, either from a good wetting of the mucoadhesive surface, or from the swelling of the mucoadhesive. In the second stage, after contact is established, penetration of the mucoadhesive into the crevices of the tissue surface or inter penetration of the chains of the mucoadhesive with those of the mucus take place.

On a molecular level, mucoadhesion can be explained based on molecular interactions. The interaction between two molecules is composed of attraction and repulsion. Attractive interactions arise from Vander walls forces, electrostatic attractions, hydrogen bonding and hydrophobic interactions. Repulsive interactions occur because of electrostatic and steric repulsion. For mucoadhesion to occur, the attractive interaction should be larger than non-specific repulsion.

Factors affecting mucoadhesion include:

1. Polymer related factors:

i) molecular weight; ii) concentration of active polymer; iii) flexibility of polymer chains; iv) spatial confirmation; and v) swelling.

2. Environment related factors:

i) pH of polymer—substrate interface; ii) applied strength; and iii) initial contact time.

3. Physiological factors:

i) mucin turns over and ii) disease state.

Systems used for the delivery of therapeutic agents via the oral route must be designed conscious of the physiology of the gastrointestinal tract. The anatomy and physiology of route of administration dictate many of the requirements for the delivery. For example, the delivery system must be able to withstand the saliva, as saliva contains digestive enzymes and other reagents for breaking down whatever is placed in the mouth. The stomach, the main digestive organ of the body, contains many digestive enzymes and has very low pH. The pH of the stomach has been measured from 1.4 to 2.1. This harsh environment causes the destruction and denaturation of proteins. The pH of the stomach changes to 4 when food is present.

Once through the harsh conditions of the stomach the delivery system reaches the small intestine, which is divided into three regions. The first region, closest to the stomach, is the duodenum, followed by the jejunum and ileum. The further down the small intestine the fewer nutrients are taken into the bloodstream. The duodenum, about 10 inches in length, composes the first 5% and the jejunum the following 40% of the length of the small intestine. The entire length of the small intestine is 5 meter and residence time within the organ typically ranges from 2-4 hr.

The lining of the small intestine are composed of the serous, muscular, areolar, and mucous layers. Only the mucous and areolar layers are the important layers with respect to drug delivery. Transport of nutrients into the body occurs through the mucous cell layer and the areolar layer where the nutrients are taken into the blood stream.

Based on the above considerations, one of ordinary skill in the art would readily devise a delivery system useful for the present invention. The present invention encompasses synthetic or naturally occurring mucoadhesive polymers that interact with the mucin and/or mucus layer covering the mucosal epithelial surface. It also encompasses the next generation mucoadhesive polymers. For example, there has been an increasing interest from researchers in targeting regions of the gastrointestinal tract using more selective molecules capable of distinguishing between the types of cells found in different areas of the gastrointestinal tract. This concept is specifically based on certain materials that can reversibly bind to cell surfaces in the gastrointestinal tract. These next generation of mucoadhesives function with greater specificity because they are based on receptor-ligand-like interactions in which the molecules bind strongly and rapidly onto the mucosal cell surface directly rather than the mucus itself. One such class of molecules that fulfill these unique requirements is lectins.

As used herein, the term “enteric coating” or “enteric coated” refers to a barrier applied to oral medication that controls the location in the digestive system where it is absorbed. Enteric coatings prevent release of medication before it reaches the small intestine. Enteric coatings work because they are selectively insoluble substances—they will not dissolve in the acidic juices of the stomach, but they will dissolve when they reach the higher pH of the small intestine. Materials used for enteric coatings include, but are not limited to, fatty acids, waxes, shellac, plastics, and plant fibers. In one embodiment, compositions of enteric coating include, but are not limited to, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate and stearic acid. One of ordinary skill in the art would readily formulate the bacteria of the present invention with any of the known enteric coating materials and methods.

In one embodiment, the present invention provides for an edible capsule form of live, non-persisting, recombinant Lactococcus lactis (L. lactis) vaccine. In one embodiment, the vaccine is produced against the highly virulent influenza virus.

Different antigen carrier systems have been proposed, including recombinant plants, bacteria, or virus-based vectors for the production and presentation of antigens (9, 16). In addition, various polymer and lipid microspheres have been used for the protection and controlled release of protein antigens in the gut. In the present invention, these two approaches have been combined to produce an oral vaccine that is effective against H5N1 infection in mice and probably in chicken as well. The recombinant L. lactis vectors were ideal to produce large quantities of antigens and deliver them orally to the gut. Considering that the gastric environment would still be somewhat hostile to L. lactis viability, an enteric-coated polymer capsule formulation of a small enough size (mm) was developed to be ingested even by mice or chickens.

In one embodiment, the enteric-coated polymer capsule formulation produces a great improvement on the overall immunogenicities of the vaccines, resulting in complete protection and survival of mice injected with an otherwise lethal dosage of H5N1 virus.

For the production and delivery of antigens, genetically engineered live vector systems have many advantages in manufacturing and processing. About 20 years ago, the plant-based vaccine development was initiated and showed the feasibility of using plants to express HBV surface proteins and viral particles to be used as vaccines (9-11). More recently, there was a study using rice grain as the delivery vehicle. The protein antigens were shown to be well protected and stably maintained in rice without requirements for refrigeration (12). Good stability of oral vaccines under ambient conditions is clearly important for the distribution of vaccines to remote areas of the world.

Alternatively, bacteria-based systems such as Salmonella, Bortedella, and Listeria spp. have also been studied extensively as antigen expression and delivery carriers. Most of them were originally pathogenic strains so they may be more immunogenic or induce stronger immune responses. In contrast, the lactic acid bacteria (LAB)-based vectors are considered safer, but may not be as immunogenic for the human immune system. Some studies have suggested the ability of certain LAB vectors to persist in the GI tract is critical for the effectiveness of vaccines. Grangette et al. conducted a direct comparison of L. Plantarum, a persisting LAB, and L. Lactis, a non-persisting LAB and found L. Plantarum to be more effective at eliciting antigen-specific immunity (22). Many other studies employed L. casei-based vectors which could also persist in human GI microbiota. However, the use of persisting bacteria might not be desirable as vehicles for edible vaccines as special consideration would be needed for biocontainment purposes (23).

The uniqueness of the present invention is the successful creation of an edible vaccine against an antigen such as influenza virus (H5N1) using the non-persisting L. lactis (24) as the carrier, loading them on mucoadhesive polymers and packaging them in enteric-coated mini-capsules. It is demonstrated that although the viability of L. lactis is rapidly diminished in the gastrointestinal tract, the antigens they carried and produced shortly after they were administered and protected temporarily by the encapsulation were sufficient to induce significant mucosal and systemic immune responses and allow all the immunized mice to survive lethal challenge of H5N1 injection.

It is possible that the vector systems themselves had specific immune stimulation effects. It has been shown that LAB can initiate inflammatory responses and activate monocytes and other antigen presenting cells (25). In the present invention, different immune responses resulted from similar vector systems that differed only in antigen expression designs were observed. The vector in which the antigens were anchored on the surface of cell wall through a PgsA domain gave the best response in every aspect. The data therefore supports the study by Poo et al. who developed the PgsA fusion system based on the N-terminal transmembrance region of the PgsBCA enzyme complex of B. subtilis (26). For avian influenza strains, the analysis of sera from mice supports the use of a neutralizing titer of ≧80 as an efficacy endpoint (27). In the present invention, the neutralization titer of capsule-L2 was 80 and the survival rate was 40% after H5N1 infection. In contrast, neutralization titers of capsule-L3 and capsule-L4 were 148 and 167, respectively, and these capsules provided 100% protection against H5N1 virus challenge. Similar plans for H5N1 challenges in chickens are in progress pending regulatory approvals.

The influenza HA antigen gene cloned in the vectors is from A/chicken/Henan/12/2004(H5N1). L2 and L3 contained the full length HA gene, while L4 contained the HA1 portion of the HA. They all induced significant humoral and systemic immune responses. Previous studies have suggested HA1 should contain almost all the HA epitopes (28-30). However, since HA1 is more prone to mutations and antigenic changes (31), it is important to design the live vector vaccines to be quickly adaptive to accommodate virus evolutions. The nisinA-induced recombinant L. lactis antigen expression vectors used herein are very flexible in design and can be optimized for antigen expression and presentation. It is also a well engineered stable system for large scale production (32, 33). The polymer mini-capsule formulation developed was also easy and inexpensive to manufacture. Although it is a simple design, the improvement in vaccine efficacy was highly significant.

In one embodiment, the present invention provides for a method of using genetically modified lactic acid bacteria such as Lactococcus lactis strains expressing the avian influenza HA gene as an oral vaccine for protection against H5N1 virus infection. In one embodiment, the oral administration of recombinant L. lactis NZ9700 (HA) microcapsules can induce significant HA-specific humoral and mucosal immune responses, and most importantly, provide protection against H5N1 virus challenge.

In one embodiment, the method comprises an oral dosing regimen which can be easily administered to both human and animal populations. In another embodiment, the method has the ability to generate a mucosal immune response.

The present invention provides a method of inducing immune responses to an antigen, comprising the step of administering to an animal or human genetically modified lactic acid bacteria expressing the antigen. Examples of lactic acid bacteria include, but are not limited to, Lactococcus, Streptococcus, Lactobacillus, Leuconostoc, Pediococcus, Brevibacterium and Propionibacterium. In one embodiment, the lactic acid bacteria are of the genus Lactococcus as described in U.S. Pat. No. 5,580,787, 6,333,188, and 7,553,956. In another embodiment, the lactic acid bacteria are of the species Lactococcus lactis.

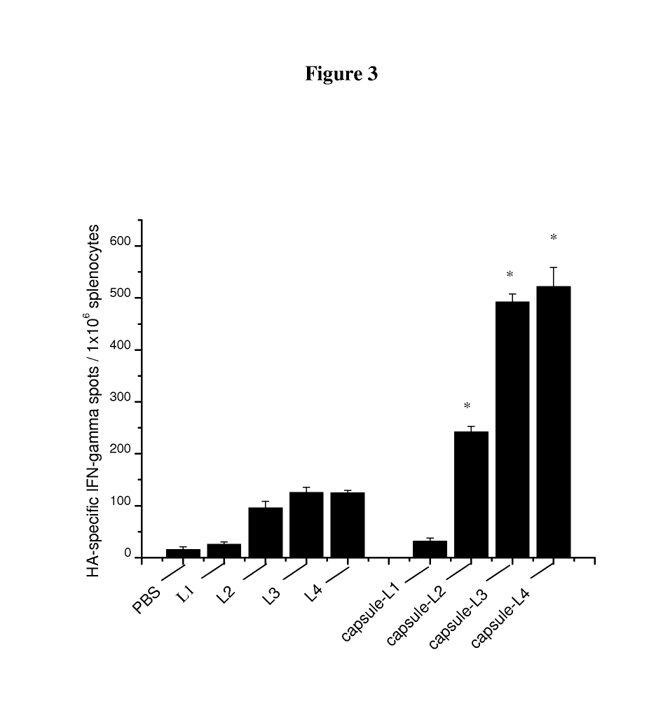

The genetically modified lactic acid bacteria of the present invention are capable of inducing immune responses when administered to a subject. Immune responses induced by the bacteria of the present invention include, but are not limited to, humoral immune responses, cellular immune responses, and mucosal immune responses. For example, the bacteria of the present invention are capable of inducing systemic IgG responses and mucosal IgA responses. In another embodiment, the bacteria of the present invention are capable of inducing cellular immune responses (see e.g. FIG. 3).

In one embodiment, the genetically modified lactic acid bacteria of the present invention are capable of inducing protective immune responses, i e immune responses that can protect immunized subjects from lethal challenges of pathogens (such as viruses or bacteria).

In general, the lactic acid bacteria of the present invention are genetically modified to express one or more antigens. In an embodiment, said antigens are heterologous. Examples of heterologous antigens include, but are not limited to, bacterial, protozoan, fungal, and viral antigens. Sources of heterologous antigens include, but are not limited to, influenza virus, helicobacter pylori, Salmonella, rotavirus, respiratory coronavirus, etc. as described in U.S. Pat. No. 6,551,830, 7,432,354, and 7,339,461.

In one embodiment, a viral antigen such as hemagglutinin of avian influenza virus H5N1 can be expressed in genetically modified lactic acid bacteria.

The genetically modified lactic acid bacteria of the present invention can be administered in amounts and by using methods that can readily be determined by persons of ordinary skill in this art. The vaccines of the present invention can be administered and formulated, for example, for oral administration, either as liquid solutions or suspensions, or solid forms suitable for solution in, or suspension in, liquid prior to administration. The preparation may also be emulsified, or the ingredients mixed with excipients such as, for example, pharmaceutical grade mannitol, lactose, starch, magnesium stearate, sodium saccharine, cellulose, magnesium carbonate, and the like. These compositions take the form of solutions, suspensions, tablets, pills, capsules, sustained release formulations, nose drops or powders.

The vaccines of the present invention can also be in the form of injectables. Suitable excipients would include, for example, saline or buffered saline (pH about 7 to about 8), or other physiologic, isotonic solutions which may also contain dextrose, glycerol or the like and combinations thereof. However, agents which disrupt or dissolve lipid membranes such as strong detergents, alcohols, and other organic solvents should be avoided. In addition, if desired, the vaccine may contain minor amounts of auxiliary substances such as wetting or emulsifying agents, pH buffering agents, and/or adjuvants well-known in the art which enhance the effectiveness of the vaccine.

Generally, the vaccine of the present invention may be administered orally, subcutaneously, intradermally, or intramuscularly in a dose effective for the production of the desired immune response. The vaccines are administered in a manner compatible with the dosage formulation, and in such amount as will be prophylactically and/or therapeutically effective. The quantity to be administered depends on the subject to be treated, the capacity of the subject's immune system to develop the desired immune response, and the degree of protection desired. Precise amounts of the vaccine to be administered in view of the subject and antigen used could be readily determined by one of skill in the art.

In one embodiment, the genetically modified lactic acid bacteria of the present invention can be administered to a subject in a number of ways, such as orally, or by intranasal administration, intramuscular injection, subcutaneous injection, and vaginal application as described in U.S. Pat. No. 7,541,044, and 7,476,686.

The genetically modified lactic acid bacteria of the present invention can be formulated in a number of ways, such as encapsulated inside acid labile microcapsules, enteric coated microcapsules and capsules, polymer hydrogels, or adhesive polymer patches. In one embodiment, the vaccine vector can be delivered by transmucosal delivery through the use of mucoadhesive polymers. Transmucosal delivery allows rapid uptake of a therapeutic agent into systemic circulation and avoids the first pass metabolism and/or some of the body's natural defense mechanism.

The present invention also provides genetically modified lactic acid bacteria expressing a heterologous antigen. Examples of lactic acid bacteria and heterologous antigens have been described above. In one embodiment, these lactic acid bacteria can be used as oral vaccines.

It should be noted that the various parameters of the present invention can be readily adjusted or substituted by one of ordinary skill in the art. For example, one of ordinary skill in the art would readily recognize that the present invention is not limited to the lactic acid bacteria or the viral antigen hemagglutinin described above. The present invention is equally applicable to using other bacteria and/or antigens known in the art. For example, the present invention can use other probiotics which are live microorganisms thought to be healthy for the host organism. Lactic acid bacteria and bifidobacteria are the most common types of microbes used as probiotics; but certain yeasts and bacilli generally known in the art are also useful. Examples of useful bacteria include, but are not limited to, Lactobacillus bulgaricus, Streptococcus thermophilus, Lactobacillus bifidus, Lactobacillus acidophilus, Lactobacillus casei, Bifidobacterium animalis, Bifidobacterium breve, Bifidobacterium infantis, Bifidobacterium longum, Escherichia coli, Lactobacillus paracasei, Lactobacillus johnsonii, Lactobacillus plantarum, Lactobacillus reuteri, Lactobacillus rhamnosus, Saccharomyces boulardii.

Moreover, the parameters of the number and size of the capsules used, the species of the mucoadhesive polymers and enteric coating employed can be readily determined and adjusted through standard experimentation. For example, any or all of these parameters can be determined or adjusted by standard in vitro or in vivo titration experiments, using various biological responses or animal survival as experimental readouts.

In one embodiment, the present invention provides a composition for inducing immune responses to an antigen, the composition comprises genetically modified bacteria that express the antigen, wherein the bacteria are formulated with mucoadhesive polymers. In one embodiment, the bacteria are lactic acid bacteria such as Lactococcus lactis. In general, the antigen can be a bacterial antigen or a viral antigen. For example, the viral antigen is hemagglutinin of avian influenza virus H5N1. In one embodiment, the mucoadhesive polymers employed are hydrophilic polymers or hydrogels. Examples of hydrophilic polymers include, but are not limited to, poly vinyl pyrrolidone, methyl cellulose, sodium carboxy methylcellulose, and hydroxy propyl cellulose. In one embodiment, the bacteria are formulated in enteric coated solid dosage forms.

The present invention also provides a method of inducing immune responses to an antigen, comprising the step of administering to a subject (such as a human or a non-human animal) the composition described above. In general, the induced immune responses include humoral immune responses, mucosal immune responses, cellular immune responses, or protective immune responses. In one embodiment, the composition is administered orally. In another embodiment, the composition is formulated in enteric coated solid dosage forms.

The present invention also provides uses of the genetically modified bacteria described herein as a medicament for inducing immune responses in a subject. In one embodiment, the medicament is administered orally. In general, the medicament can be applied for uses in subject such as human, animal, fish, bird, and veterinary uses.

The present invention also provides a genetically modified lactic acid bacteria expressing a heterologous antigen, wherein the bacteria are formulated with mucoadhesive polymers. Examples of mucoadhesive polymers have been described above. In one embodiment, the lactic acid bacteria are of the genus Lactococcus. In another embodiment, the lactic acid bacteria are of the species Lactococcus lactis. In general, the heterologous antigen can be a bacterial antigen or a viral antigen. In one embodiment, the heterologous antigen is hemagglutinin of avian influenza virus H5N1. In another embodiment, the present invention also provides a composition comprising the above-described genetically modified lactic acid bacteria that express a heterologous antigen.

The present invention also provides a kit for inducing immune responses in a subject, wherein the kit comprises the genetically modified bacteria as described herein.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains.

### Example 1

### Materials and Methods

Recombinant L. lactis vectors

Three different antigen expressing plasmids were constructed and named as pNZ8150-HA, pNZ8110-HA and pNZ8110-pgsA-HA1. The pNZ8148 plasmid was purchased from Netherlands NIZO. The plasmids were transformed into the L. lactis NZ9000 stains by electroporation. The most highly expressed clones were selected and cultured at 30° C. in media based on M17 medium supplemented with 0.5% (wt/vol) glucose. Chloramphenicol was used at a concentration of 10 μg/ml.

### Western Blot Analysis and Immunofluorescence Microscopy

The antigen expressions were induced in all the recombinant L. lactis strains by adding nisinA to the final concentration of 10 ng/ml. Growth was continued for 3 hours. For the L1, L2, L4 cultures, L. lactis cells were harvested, washed three times with 500 u1 sterile phosphate-buffered saline (PBS), and resuspended. Aliquot of the samples were mixed with 6× loading buffer and boiled 10 minutes. Extracts were run on SDS-PAGE (10% acrylamide) and transferred to polyvinylidene difluoride membrane (PVDF, Millipore, USA). Antibody reactions and detection (enhanced chemiluminescence) were performed according to the manufacture's recommendations (Amersham Bioscience, Sweden). For the L3 culture, the supernatant was collected for the western blot analysis. For the immunofluoresence staining, the L4 L. lactis cells were harvested after induction, and incubated with polyclonal mouse anti-HA antibody at 4° C. for 30 minutes followed by fluorescein isothiocyanate (FITC)-conjugated anti-mouse IgG. The resulting cell pellets were examined using an Olympus fluorescence microscope.

Preparation of the Enteric Coated Capsule Formulation of L. lactis

Recombinant L. lactis was prepared into solutions with the concentration of 10¹¹ colony-formation units (CFU)/ml. 10 μl of the recombinant L. lactis solution containing 1×10⁹ CFU was mixed with 0.5 mg of BSA and methyl cellulose (MC), air dried, and packaged into enteric coated capsules. (mini-capsule, each about 4×1 mm)

Analysis of L. lactis Viability in Simulated Gastric and Intestine Fluid

The capsules were immersed in simulated gastric fluid at pH1.0 with low speed agitation for two hours and then dropped into phosphate.—buffer at pH 6.8 for 45 minutes to release the encapsulated contents. Viable cells were counted by gradient dilution methods.

### Animals and Animal Immunizations

Six-week-old female BALB/c mice were purchased from China SLC, Shanghai, China. The mice were housed in the specific pathogen-free (SPF) Animal Center of Shanghai Jiao Tong University. Before each dose, they were fasted for 6 hours and then administer 10 ul of the recombinant L. lactis solution or 1 capsule using a 21-gauge feeding tube. Immunizations were repeated at 2, 4, 6 weeks after the initial dosing. L4 was representatively chosen for the chicken experiments, seven-day-old female SPF chickens were inoculated by subcutaneous injection and oral immunization at the doses of 10⁸CFU/chicken Immunizations were given at days 1 and 2, 7, 14, 21, 28, 29.

### ELISA Assay

Sera from mice or chickens were collected 10 days after the last dosing. HA-specific antibody responses were detected by avidin-biotin system (ABS)-enzyme-linked immunosorbent assay (ELISA) (19). 10 μg/ml Influenza A virus (A/chicken/Henan/12/2004(H5N1)) recombinant HA protein was used to coat 96-well microplates. At the same time, Fecal pellets (50 mg) for each group of six mice were collected and suspended in 250 μl sterile PBS, centrifuged down at 15 000× rpm for 10 minutes, and the supernatants tested for IgA by indirect ELISA. The mean antibody titer was expressed as the highest dilution that yielded an optical density greater than twice the mean plus one standard deviation of that of similarly diluted negative control samples.

### IFN-γ ELISpot Assay

The IFN-γ ELISpot assay was performed one week after the final immunization using an ELISpot kit for mouse IFN-γ as recommended by the manufacturer (R&D Systems, USA). Briefly, mouse IFN-γ microplate was added 200 μl/well of sterile culture media and incubated for 20 minutes at room temperature. After aspirating the culture media from the wells, the plates were added 100 μl of 1×10⁶ splenocytes per well. 10 μg/ml of HA-specific peptide (ISVGTSTLNQRLVP) was used as stimuli for 48 hours in a humidified 37° C. CO₂ incubator. Control wells were not stimulated with HA-specific peptide. After incubation, each well was aspirated and washed, the plates were treated sequentially with biotinylated anti-mouse IFN-γ antibody, alkaline phosphatase conjugated stretavidin and the substrate solution to reveal the spots. The developed microplate could be analyzed by counting spots using a dissection microscope.

### Neutralization Assay

Determination of endpoint neutralizing antibody titers was performed by microneutralization assay, as previously described (20). Briefly, serial 2-fold dilutions of sera treated with receptor-destroying enzyme (RDE) from Vibro cholerae were mixed and incubated with 35 μl 100 50% tissue culture infective doses (TCID₅₀) of H5N1 virus, then added to Madin-Darby canine kidney (MDCK) cells and incubated for 1 h. The H5N1 virus-infected MDCK cells were further cultured for 72 h at 37° C. in the presence of 5% CO2, and the neutralizing titer was determined by hemagglutination test. For the HA test, 50 μl of 0.5% cock red blood cells was added to 50 μl of cell culture supernatant and incubated at room temperature for 30 min. The TCID₅₀ was determined on the basis of the Reed-Muench method (21). The neutralization titer (IC₅₀) was defined as the reciprocal of the antiserum dilution at which H5N1 virus entry was 50% inhibited.

### H5N1 Virus Challenge

For the challenge experiment, mice were anesthetized and intranasally challenged with 20 μl 10×50% lethal dose (LD₅₀) H5N1 virus two weeks after the last immunization. After infection, the mice were weighed and monitored for signs of illness for 14 days. The challenge experiments were strictly performed under biosafety level-3-plus enhancement conditions.

### Statistical Analysis

Statistical analysis of the experimental and control data were performed by one-way factorial analysis of variance. P-values less than 0.05 were considered as a statistical significance.

### Example 2

### Recombinant Lactococcus lactis Vectors

The construction of recombinant L. lactis vectors expressing the Influenza H5N1 HA antigen

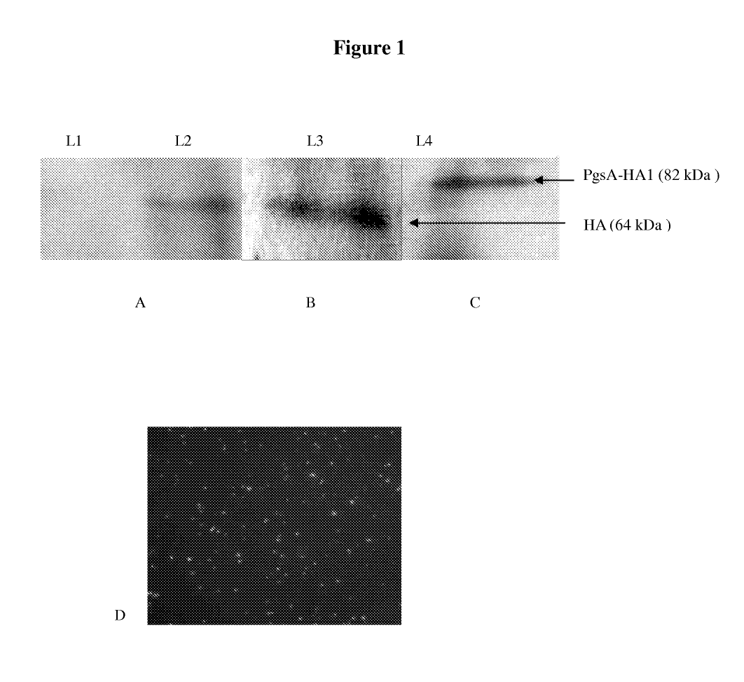

Four different L. lactis vectors were constructed and named L1, L2, L3, and L4. The vector descriptions and the specific HA expression plasmid contained in each vector were listed in Table 1. Western blot analysis showed the L1 as a control vector which doesn't express any HA (FIG. 1A), and L2 expressed the HA protein (64 kDa) which was mostly found in the cell lysate (FIG. 1A). L3 had an usp45 signal sequence cloned before the HA gene; so expressed HAs were mostly found in the culture supernatant (FIG. 1B). L4 contained a plasmid encoding the HA1 protein (38 kDa) fused with the PgsA surface display motif (44 kDa). The resulted protein expressed had 82 kDa MW (FIG. 1C). In addition, the cell wall anchored distribution of the HA1 protein (L4) was confirmed by immunofluorescence staining (FIG. 1D).

Enteric Capsule Preparation and L. lactis Release in Simulated Gastrointestinal (GI) Environment

About 10⁹ CFU of the recombinant L. lactis vectors were packaged in an enteric capsule (mini-capsule), each measuring about 4×1 mm in size. The samples were named capsule-L1, capsule-L2, capsule-L3, and capsule-L4. The capsule samples as well as solution samples were treated with simulated gastric fluid at pH 1.0 for 2 hour and then released in simulated intestinal buffer at pH 6.8 for 45 minutes. The resulted live L. lactis cell counts (CFU) were listed in Table 2. The capsule groups showed strong acid resistance and survived at pH 1.0, while in the solution groups the live vector counts showed massive loss of viability (>20000-fold) after the treatments.

### HA-Specific IgG and IgA Responses After Oral Administration

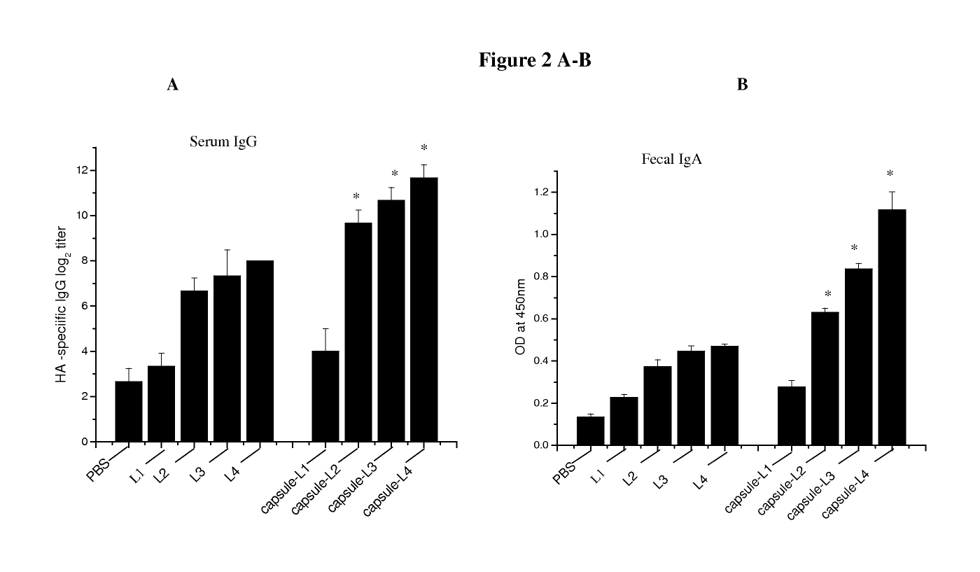

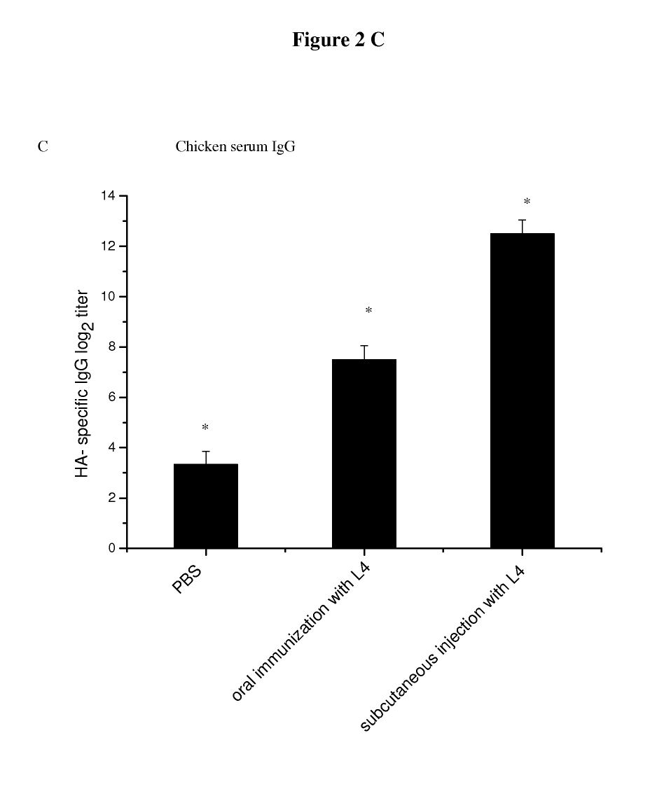

The mice were immunized four times at week 0, 2, 4, 6 by oral administration of the recombinant L. lactis solutions or enteric capsules. HA-specific serum IgG and fecal IgA levels were measured 10 days after the last immunization dosing. The mean log₂ titers of serum IgG of all the tested groups were shown in FIG. 2A. All the HA expressing vectors (solution and capsules) resulted in significant production of HA-specific serum IgG, while PBS and L1 samples did not. In general, encapsulated groups had higher titers than the solution groups. The group dosed with capsule-L4 reached the highest antibody titer. To examine the HA-specific mucosal immune response, the production of mucosal IgA antibody was examined using fecal pellets (FIG. 2B). Again, the enteric capsule groups all developed significant IgA antibody responses. The capsule-L4 gave the best results. In experiments where seven-day-old chickens were inoculated either by subcutaneous injection or by oral administration at days 1 and 2, 7, 14, 21, 28, 29, sera were analyzed 10 days after the final immunization. Both delivery mechanisms yielded significant serum IgG titers, although chickens treated by subcutaneous injection were somewhat higher than the orally immunized groups (FIG. 2C).

### Neutralizing Antibody Titers in Mice

The neutralizing antibody titers in each treatment groups were measured using the microneutralization (MN). Neutralization titers values of the enteric capsule groups (capsule-L2, capsule-L3 and capsule-L4) were all higher than 80, suggesting good neutralization activities of the antibodies generated (Table 3).

### Antigen Specific T Cell Responses

The IFN-γ ELISpot assay was also used to examine the HA-specific T cell response resulted from recombinant L. lactis after oral administration. Splenocytes (10⁶ cells) from each treatment group were collected and stimulated with 10 μg/ml of HA epitope peptide (ISVGTSTLNQRLVP). The resulted IFN-γ expressing T cell numbers were counted and plotted in FIG. 3. Again, there were HA-specific T cell responses generated by oral administration of enteric coated recombinant L. lactis . The encapsulated groups (capsule-L2, capsule-L3 and capsule-L4) were much more effective than the respective solution groups.

### H5N1 Virus Challenge Experiment

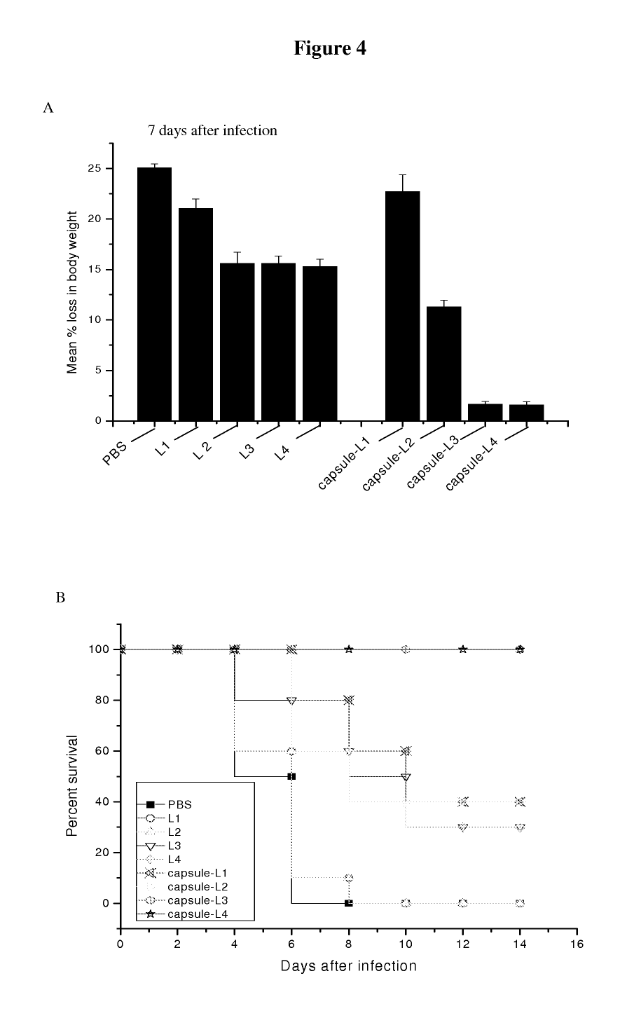

Two weeks after the final immunization, the mice were intranasally challenged with lethal doses of highly pathogenic H5N1 viruses and closely monitored for 14 days for weight loss and mortality. After viral challenge, all mice experienced certain levels of body weight loss (FIG. 4A), but mice immunized with capsule-L3 and capsule-L4 gradually recovered after 8 days and 100% survival. In contrast, the naïve mice (PBS treated) and mice immunized with the empty plasmid vector all died within 10 days of challenge (FIG. 4B).

TABLE 1
Recombinant Lactococcus lactis vectors

| | | | | Antigen |
|---|---|---|---|---|
| Vector | Host Starin* | Plasmid | Antigen | location |
| L1 | Lactococcus | pNZ8110 | | |
| | lactis | (pNZ8148 with an | | |
| | NZ9000 | usp45 signal sequence) | | |
| L2 | Lactococcus | pNZ8150-HA | HA | cytoplasm |
| | lactis | (HA gene cloned in | | |
| | NZ9000 | E. coli-L. lactis shuttle | | |
| | | vector pNZ8148) | | |
| L3 | Lactococcus | pNZ8110-HA | HA | secreted |
| | lactis | (HA gene cloned | | |
| | NZ9000 | in pNZ8110) | | |
| L4 | Lactococcus | pNZ8110-PgsA-HA1 | HA1 | cell wall |
| | lactis | PgsA-HA1 fusion gene | | anchored |
| | NZ9000 | clone in pNZ8110) | | |

(*Lactococcus lactis NZ9000: engineered stain based on MG1363 with the nisin inducible expression cassette containing nisR and nisK genes)

TABLE 2
The relative live L. lactis count after
simulated gastric fluid treatment

| L. lactis vector | live bacteria number (CFU) |
|---|---|
| L1 | 3.5 × 10⁴ |
| L2 | 3.6 × 10⁴ |
| L3 | 3.8 × 10⁴ |
| L4 | 3.6 × 10⁴ |
| capsule-L1 | 10⁹ |
| capsule-L2 | 10⁹ |
| capsule-L3 | 10⁹ |
| capsule-L4 | 10⁹ |

TABLE 3
Neutralization assay of H5N1 virus with immune serum

| | Neutralization |
|---|---|
| Mice orally immunized with | titer of serum (IC₅₀) |
| PBS | <10 |
| L1 | <10 |
| L2 | 47 |
| L3 | 68 |
| L4 | 70 |
| capsule-L1 | <10 |
| capsule-L2 | 80 |
| capsule-L3 | 148 |
| capsule-L4 | 167 |

### Example 3

### Further Refinement

To adjust or lower the dose of bacteria used in the present invention, standard in vitro or in vivo titration experiments can be done, using various biological responses or animal survival described above as experimental readouts

The same kind of titration experiments can also be done to determine the effects of the size of the coated capsule on immune response induction. Capsules of various size can be tested in the in vitro or in vivo experiments described above to investigate the effects on the induction of cellular immune responses, mucosal immune responses, and/or protective immune responses.

Similarly, the same kind of titration experiments can also be done to determine the effects of using different coating material or mucoadhesive polymers. Capsules coated with different coating materials, or bacteria formulated with different mucoadhesive polymers can be tested in the in vitro or in vivo experiments described above to investigate the effects on the induction of cellular immune responses, mucosal immune responses, and/or protective immune responses.

### REFERENCES

1. Subbarao, K., Klimov, A., Katz, J., Regnery, H., Lim, W., Hall, H., Perdue, M., Swayne, D., Bender, C., Huang, J., Hemphill, M., Rowe, T., Shaw, M., Xu, X., Fukuda, K. & Cox, N. (1998) Science 279, 393-396.
2. Johansson, B. E., Brett, I. C., Focosi, D. & Poland, G. A. (2006) N. Engl. J. Med. 354, 2724-2725.
3. Kilbourne, E. D., Smith, C., Brett, I., Pokorny, B. A., Johansson, B. & Cox, N. (2002) Proc. Natl. Acad. Sci. USA 99, 10748-52.
4. Wei, C. J., Xu, L., Kong, W. P., Shi, W., Canis, K., Stevens, J., Yang, Z. Y., Dell, A., Haslam, S. M., Wilson, I. A. & Nabel, G. J. (2008)J. Virol. 82, 6200-6208.
5. Chen, M. W., Cheng, T. J. R., Huang, Y., Jan, J. K., Ma, S. H., Yu, A. L., Wong, C. H. & Ho, D. D. (2008) Proc. Natl. Acad. Sci. USA 105, 7235-7240.
6. Kong, W. P., Hood, C., Yang, Z. Y., Wei, C. J., Xu, L., Garcia-Sastre, A., Tumpey, T. M. & Nabel, G. J. (2006) Proc. Natl. Acad. Sci. USA 103, 15987-15991.
7. Hoelscher, M., Garg, S., Bangari, D. S., et al. (2006) Lancet 367, 475-481.
8. Gao, W., Soloff, A. C., Lu, X., Montecalvo, A., Nguyen, D. C., Matsuoka, Y., Robbins, P. D., Swayne, D. E., Donis, R. 0., Katz, J. M., Barratt-Boyes, S. M. & Gambotto, A. (2006)J. Virol. 80, 1959-1964.
9. Mason, H. S., Lam D. M. K. & Amtzen, C. J. (1992) Proc. Natl. Acad. Sci. USA 89, 11745-11749.
10. Lam, D. M. K. & Shi, J. (1996) Agro-Food-Industry high-tech March/April, 7-12.
11. Lam, D. M. K., Shi, J. & Zhang, X. Y. (1997) Foreign Medical Sciences China 20, 145-149.
12. Nochi, T., Takagi, H., Yuki, Y., Yang, L. J., Masumura, T., Mejima, M., Nakanishi, U., Matsumura, A., Uozumi, A., Hiroi, T., Morita, S., Tanaka, K., Takaiwa, F. & Kiyono, H. (2007) Proc. Natl. Acad. Sci. USA 104, 10986-10991.
13. Xin, K. Q., Hoshino, Y., Toda, Y., Igimi, S., Kojima, Y., Jounai, N., Ohba, K., Kushiro, A., Kiwaki, M., Hamajima, K., Klinman, D. & Okuda, K. (2003) Blood 102, 223-228.
14. Robinson, K., Chamberlain, L. M., Schofield, K. M., Wells, J. M. & Page, R. W. F. (1997) Nat. Biotechnol. 15, 653-657.
15. Gilbert, C., Robinson, K., Page, R. W. F. & Wells, J. M. (2000) Infect. Immun. 68, 3251-3260.
16. Cho, H. J., Shin, H. J., Han, I. K., Jung, W. W., Kim, Y. B., Sul, D. & Oh, Y. K. (2007) Vaccine 17, 607-616.
17. Klijn, N., Weerkamp, A. H. & de Vos, W. M. (1995) Appl. Environ. Microbiol. 61, 2771-2774.
18. Gruzza, M., Fons, M., Ouriet, M. F., Duval-Iflah, Y. & Ducluzeau, R., (1994) Microb. Releases 2, 183-189.
19. Katz, J. M., Lu, X., Young, S. A.& Galphin, J. C. (1997)J. Infect. Dis. 175, 352-363.
20. Rowe, T., Abernathy, R. A., Hu-Primmer, J., Thompson, W. W., Lu, X. H., Lim, W., Fukuda, K., Cox, N. J. & Katz, J. M. (1999)J. Clin. Microbiol. 37, 937-943.
21. Röhm, C., Horimoto, T., Kawaoka, Y., Süss, J. & Webster, R. G (1995) Virology 209, 664-670.
22. Grangette, C., Muller-Alouf, H., Geoffroy, M. C., Goudercourt, D. & Turneer, M. (2002) Vaccine 20, 3304-3309.
23. Steidler, L., Neirynck, S., Huyghebaert, N., Snoeck, V. & Vermeire, A. (2003) Nat. Biotechnol. 21, 785-789.
24. Kimoto, H., Nomura, M., Kobayashi, M., Mizumachi, K.& Okamoto, T. (2003) Can. J. Microbiol. 49, 707-711.
25. Mercenier, A., Pavan, S. & Pot, B. (2003) Curr. Pharm. Des. 9, 175-191.
26. Poo, H., Pyo, H. M., Lee, T. Y., Yoon, S. W., Lee, J. S., Kim, C. J., Sung, M. H. & Lee, S. H. (2006) Int. J. Caner 119, 1702-1709.
27. Eichelberger, M., Golding, H., Hess, M., Weir, J., Subbarao, K., Luke, C. J., Friede, M.& Wood, D. (2007) Vaccine 26, 4299-4303.
28. Jeffery, K. & Taubenberger, J. K. (1998) Proc. Natl. Acad. Sci. USA 95, 9713-9715.
29. Shih, A. C. C., Hsiao, T. C., Ho, M. S. & Li, W. H. (2007) Proc. Natl. Acad. Sci. USA 104, 6283-6288.
30. Suzuki, Y., Ito, T., Suzuki, T., Holland J. R. E., Chambers, T. M., Kiso, M., Ishida, H. & Kawaoka, Y. (2000)J. Virol. 74, 11825-11831.
31. Stevens, J., Corper, A. L., Basler, C. F., Taubenberger, J. K., Palese, P. & Wilson, I. A. (2004) Science 303, 1866-1870.
32. de Ruyter, P. G., Kuipers, 0. P. & de Vos, W. M. (1996) Appl. Environ. Microbiol. 62, 3662-3667.
33. Bahey-El-Din, M., Casey, P. G., Griffin, B. T. & Gahan, C. G. (2008) Vaccine 26, 5304-5318.

What is claimed is:

1. A composition for inducing immune responses to an antigen, the composition comprises genetically modified bacteria that express the antigen, wherein the bacteria are formulated with mucoadhesive polymers.

2. The composition of claim 1, wherein the bacteria are lactic acid bacteria.

3. The composition of claim 2, wherein the lactic acid bacteria are of the genus Lactococcus.

4. The composition of claim 2, wherein the lactic acid bacteria are of the species Lactococcus lactis.

5. The composition of claim 1, wherein the antigen is a bacterial antigen or a viral antigen.

6. The composition of claim 5, wherein the viral antigen is hemagglutinin of avian influenza virus H5N1.

7. The composition of claim 1, wherein the mucoadhesive polymers are hydrophilic polymers or hydrogels.

8. The composition of claim 7, wherein the hydrophilic polymers are selected from the group consisting of poly vinyl pyrrolidone, methyl cellulose, sodium carboxy methylcellulose, and hydroxy propyl cellulose.

9. The composition of claim 1, wherein the bacteria are formulated in enteric coated solid dosage forms.

10. A method of inducing immune responses to an antigen, comprising the step of administering to a subject the composition of claim 1, wherein the composition comprises bacteria expressing the antigen and the bacteria are formulated with mucoadhesive polymers.

11. The method of claim 10, wherein the subject is a human or a non-human animal.

12. The method of claim 10, wherein the bacteria express hemagglutinin of avian influenza virus H5N1.

13. The method of claim 10, wherein the immune responses are humoral immune responses, mucosal immune responses, cellular immune responses, or protective immune responses.

14. The method of claim 10, wherein the composition is administered orally.

15. The method of claim 10, wherein the composition is formulated in enteric coated solid dosage forms.

16. The composition of claim 1 for use as a medicament for inducing immune responses to an antigen in a subject.

17. The composition of claim 16, wherein the medicament is administered orally.

18. The composition of claim 16, wherein the composition comprises bacteria expressing hemagglutinin of avian influenza virus H5N1

19. A genetically modified lactic acid bacteria expressing a heterologous antigen, wherein the bacteria are formulated with mucoadhesive polymers.

20. The bacteria of claim 19, wherein the lactic acid bacteria are of the genus Lactococcus.

21. The bacteria of claim 19, wherein the lactic acid bacteria are of the species Lactococcus lactis.

22. The bacteria of claim 19, wherein the heterologous antigen is a bacterial antigen or a viral antigen.

23. The bacteria of claim 19, wherein the heterologous antigen is hemagglutinin of avian influenza virus H5N1.

24. A composition comprising the bacteria of claim 19.
